(19) United States
(12) Patent Application Publication
(10) Pub. No.: US 2003/0112409 A1
(43) Pub. Date: Jun. 19, 2003

(54) Cataract prescreening systems and methods

(76) Inventors: Mary Jo Vaughn, Garden Grove, CA (US); Andrew J. Blahut, Garden Grove, CA (US); Luis M. Ortiz, Dallas, TX (US)

Correspondence Address: Luis M. Ortiz, Registerd Patent Attorney, P.O. Box 7763, Dallas, TX, 75209, US

(21) Appl. No.: 10/273,471

(22) Filed: Oct. 18, 2002

Related U.S. Application Data
(60) Provisional application No. 60/330,388, filed on Oct. 18, 2001.

(51) Int. Cl. A61B 3/00

(52) U.S. Cl. 351/200

(57) ABSTRACT

Methods and systems for prescreening a patient of ocular disease are disclosed. One method of corneal analysis includes steps of providing blue light from a light source into the corneal area of a patients eye and assessing the illuminated corneal area of a patients eye to detect the presence of cataract material. Another method of corneal analysis, includes steps of providing blue light from a light source into the corneal area of a patients eye, recording illumination of the corneal area and assessing recorded illumination of the corneal area to detecting the presence of cataract material. An apparatus is described for analyzing the corneal area of a patients eye including an illumination source for providing blue light into the corneal area of a patients eye. An apparatus is also described for assisting an evaluation of disease in a patient's eye that includes blue light illumination source for illuminating the corneal area of the eye, imaging means for recording illumination of a the eye and an imaging area for providing a controlled environment for illumination and imaging of the corneal area of the eye. Also described is the combination of an apparatus for prescreening for cataracts with a glaucoma assessment apparatus.

This application claims priority to Provisional Patent Application, Ser. No. 60/330,388, Filed Oct. 18, 2001, for “Cataract Prescreening Systems and Methods.”

### TECHNICAL FIELD OF THE INVENTION

The present invention generally relates to noninvasive determination of disease presence within the eye of a patient. More particularly, the present invention relates to prescreening methods and apparatuses for non-invasively determining the presence of an ocular disease.

### BACKGROUND

A person with a mature cataract, which significantly impairs visual function, can generally be treated by surgically extracting the implanted lens and corneal material within the anterior chamber of a patient's eye and replacing the impaired lens with either an intraocular lens or an extraocular lens. The cataract condition, however, cannot be addressed until properly diagnosed or determined. Furthermore, it is foreseeable with rapid development in the field of biotechnology that early intervention will enable new pharmaceutical treatments to be administered. Treatment may not be as invasive with such advances in biotechnology and improved vision may be restored to a patient sooner if a cataract condition can be discovered early.

Many different methods and apparatuses have been developed in the past to help determine the existence or extent of cataract disease. These methods and apparatus have generally made the determination based either on visual acuity tests or on an analysis of light exiting the eye of the patient. These may not be optimum indicators of a cataract, however, due to various anomalies. In the case of visual acuity tests, that depend upon light reaching the retina, the use of high contrast letters or figures may enable the patient to recognizes the letters and figures and thus “pass” the visual acuity test regardless of a cataract condition.

Similarly, in another test that compares a photograph of a person's lens to a standardized series of photographs of a lens with different degrees of cataract formation in different parts of the lens, the resulting photographic images depend upon back-scattered light from the lens. Because the back scattered light may not correlate highly with the location of the cataract and what the patient sees, a clinician using the photographs as the basis of an analysis will not be able to accurately determine the effect of opacities upon the patient's visual function and accordingly the patient may “pass” or may “fail” the test incorrectly. Moreover, in U.S. Pat. No. 4,863,261, issued to J. Flammer, entitled “Method of and Apparatus for Measuring the Extent of Clouding of the Lens of a Human Eye,” light exiting the eye, i.e. “back scattered” light, is analyzed with respect to incident radiation to determine the extent of clouding of the lens.

Benedek et al., in U.S. Pat. No. 4,993,827 for “Method for Detecting Cataractogenesis”, issued Feb. 19, 1991, collect and determine the intensity of light scattered from a measurement location in the lens and compares this value to the intensity of light scattered by a normal, clear lens to determine the degree of cataractogenesis at the specific measurement location.

Taratuta et al., in U.S. Pat. No. 5,072,731 for “Apparatus for Detecting Cataractogenesis Using Quasielastic Light Scattering”, issued Dec. 17, 1991, analyze the light scattered from the lens using an autocorelation function or the power spectrum to separate the light fluctuation into two components: one caused by fast diffusing proteins and one caused by slow diffusing protein aggregates. The data is then compared to reference curves to determine the degree of cataractogenesis.

In each of the above back scattering techniques, low intensity light must be incident upon the eye in order to avoid damage to the eye. Of the low intensity incident light, a portion thereof is reflected for analysis. Because of the limited incident intensity, only a small amount of light is reflected back to a photomultiplier of limited quantum efficiency for measurement. The limited amount of reflected light and limited quantum efficiency of the photomultiplier make accurate analysis difficult.

Kandel, et al, in U.S. Pat. Nos. 5,609,159 and 5,908,39 entitled “Method and apparatus for noninvasive determination of a disease state of a human eye,” issued Mar. 11, 1998 and Jun. 1, 1999, respectively, each describe techniques for an improved, noninvasive, ocular disease state determination by assessing the light that reaches the patient's retina and forms the proximal stimulus that the patient's visual system uses in the first stage of the perceptual process. The through-put quality of the axial portion of the lens is thereby measured indirectly by using the patient's visual system as a visual null indicator that enables one to track the rate of cataract formation. Use of the patient's retina itself as the detector provides a system of inherently superb quantum efficiency in contrast to that of known photomultipliers.

In Hanaki U.S. Pat. No. 6,074,063 entitled “ophthalmic apparatus for photographing an anterior part of an eye,” issued Jun. 13, 2000, an apparatus is disclosed comprising a sectional image photographing optical system for photographing a sectional image of the anterior part of the eye, a rotating device for rotating the sectional image photographing optical system, a retroillumination image photographing optical system for photographing a retroillumination image of the anterior part of the eye, a determining device for obtaining a rotation angle that the sectional image photographing optical system is to be rotated by the rotating device based on the retroillumination image photographed by the retroillumination photographing optical system and a controlling device for controlling operation of the rotating device based on the rotation angle obtained by the determining device.

As shown by the prior art, use of the patient's own retina as a detector enables elaborate instruments and a methods employing light to assess the disease state of a patients eye. Oftentimes, assessments such as those described are undertaken when the disease has already become an obvious annoyance or danger to the patient. What is needed are methods and systems that enable prescreening of, for example, cataract disease so that treatment can be provided before the disease is at an advanced state.

### SUMMARY OF THE INVENTION

The present inventors have recognized that prescreening for cataracts can and should be provided utilizing less sophisticated or elaborate methods and systems that are used only after cataract disease has already grown to an obvious state.

Briefly, the present invention satisfies the need for early intervention and prescreening by providing apparatuses and methods that enable preliminary ocular disease identification based on blue light entering into the corneal area (i.e., anterior chamber region and lens) of a patient's eye.

In accordance with the above, it is a feature of the present invention to provide a noninvasive method for identifying ocular disease through prescreening methods.

It is another feature of the present invention to utilize a hand held light emitting apparatus to direct blue light into a patients eye in order to prescreen for the existence of cataract disease.

It is yet another feature of the present invention to provide a method and an instrument to assess the precursor to cataract formation in the eye of a patient in combination with other ocular prescreening methods and systems, such as technology presently available for glaucoma testing.

The present invention can provide, in a first aspect, a method for prescreening a patient for the existence of cataract disease in an eye of a patient. The method comprises providing blue light from a blue light source into the corneal area of a patient's eye, observing the patient's corneal lens in the presence of the light to determine if material within the corneal area is illuminating in the presence of the light. If illuminated material has been identified, the patient can be referred for cataract treatment. If no material has been identified, the patient passes the cataract prescreening process.

The present invention can provide, in a second aspect, an apparatus for assisting in the identification of disease in an eye of a patient. The apparatus comprises a housing containing a blue light source. The apparatus may be a portable, hand held device. If hand held, the device may be battery operated or powered by an ac power cord.

In another embodiment of the present invention, an apparatus for assisting in the identification of disease in an eye of a patient may be incorporated within a housing including a camera for recording the identification of illuminated material within the retinal area of a patients eye.

In another embodiment of the present invention, an apparatus for assisting in the identification of disease in an eye of a patient may be included in the housing of a glaucoma testing apparatus. In combination, such an apparatus would provide practitioners the ability to un-invasively assess a patient for multiple diseases.

These, and other aspects, features and advantages of this invention will become apparent from the following detailed description of the various aspects of the invention taken in conjunction with the accompanying drawings.

### BRIEF DESCRIPTION OF THE DRAWINGS

FIG. 1 illustrates basic components of a cataract prescreening apparatus;

FIG. 2 illustrates a hand held cataract prescreening system in accordance with a preferred embodiment of the present invention including illustration of a light source providing light entering an eye of a patient;

FIG. 3 illustrates a cataract prescreening system including blue light source and a camera for recording cataract status;

FIG. 4 illustrates a combined cataract and glaucoma prescreening systems; and

FIG. 5 is a flow diagram of methods steps for prescreening a patient for ocular disease using the system of the present invention.

### DETAILED DESCRIPTION OF PREFERRED EMBODIMENTS

The colors of light that the human eye is able to see range roughly from red to blue in color. Blue light has a higher frequency (more energy) than red light. The light that has frequency just lower than red light is called “infra-red”, and the light that has frequency just higher than blue light is called “ultra-violet”. Both infrared and ultra violet-light is beyond our eyes range for efficiently detecting, however they are still very important.

A “black light” is generally known as a light bulb or light source designed to emit ultra-violet light. The reason these illumination sources are called “black” is that if you look at the actual bulb, it does not seem very bright (sort of a dim violet color), and if you put a black light in a dark room it really does not brighten it very much . . . the room remains almost black. These bulbs do emit much light, however, it's just that the human eye can't see it.

Some materials have special properties that enable them to absorb ultra-violet light and then re-emit the light at lower frequencies that our eyes can see. This is called “fluorescence”. Such materials are oftentimes found on our t-shirts, jackets or shoes, and when one walks near a black-light the materials will seem to “glow” since they are translating the invisible ultra-violet light into easy to see colors, most often white. The general feeling to human perception is that illuminated objects have a deep tint of blue.

With reference to FIG. 1, basic components of a cataract prescreening apparatus 10 are illustrated. A prescreening apparatus 10 includes at least one light source 11, a switch 15 and power source 12. These components will normally being located within a housing 13. The light source 11 can be in the form of an LED, light bulb or low powered, scattered laser. Finally, an electrical power source 12 can be provided in the form of a portable (DC battery) or residential/commercial (AC) sources.

In accordance with the present invention, it is preferred that light emanating from the light source should be perceived to be blue in color. What will hereinafter be referred to as “Blue light,” but which can also referred to, and is generally known as, “black light,” is also best known for its enhanced illumination of fluorescent material. The use of blue light in the present invention has also been found by the present inventors to be effective in the illumination of cataract material within the corneal area (i.e., anterior chamber region, lens) of the human eye.

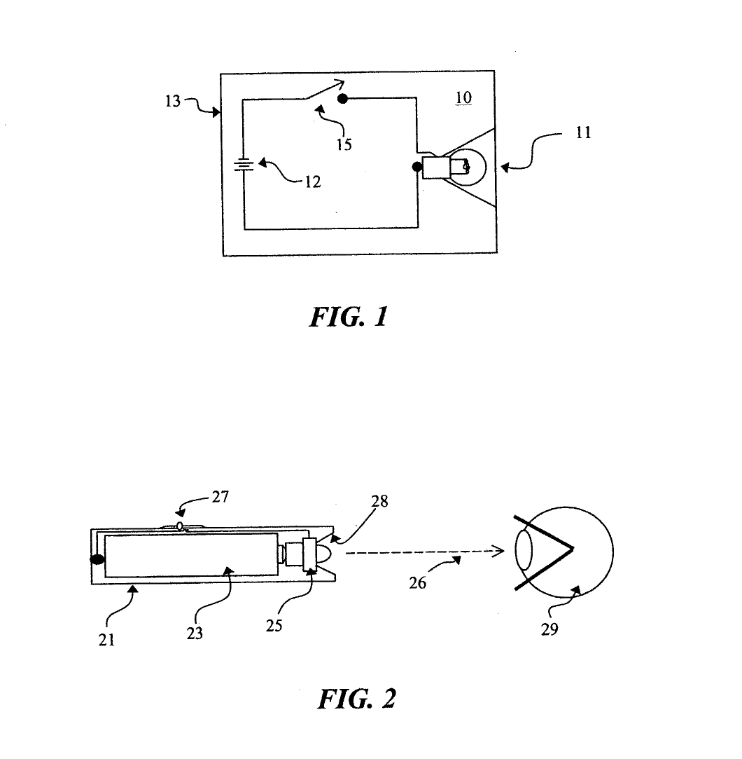

Referring to FIG. 2, a first embodiment of the present invention is illustrated. The cataract prescreen apparatus can be provided in the form of a hand held, portable device. Such a device can take the form of a penlight or flash light. As known in the art of such devices, the prescreen apparatus will have a housing 21, a battery power source 23, switch 27, and illumination source 25. Unlike typical flashlights or penlights, a portable device that is configured for cataract screening will have an illumination source 25 that provides blue light. Blue light can be focused in the direction of a patients eye 29 with a conical reflector 28, also known in the flashlight art. The battery power source 23 can be rechargeable or be provided in the form of commercially available batteries (e.g., AA, AAA, C, D, etc.).

Referring to FIG. 3, another embodiment of the present invention is illustrated. An apparatus for prescreening cataract patients can be provided as desktop, countertop device, and/or handheld device that further provides imaging technology to record a patient's condition. The housing of the prescreening apparatus 30 includes at least one illumination source 33 and a camera 35. An imaging area 36 is provided and should be configured to allow a patient to look into a controlled environment within the imaging area. Illumination source 33 illuminates the patients corneal area (including the lens) of the eye and camera 35 can capture an image of the patient's cornea area. If cataract material is existent within the corneal area, blue light from the illumination source should reveal it and the camera should capture it for further evaluation. If no cataract material is existent in the patients corneal area, imaging will be unremarkable.

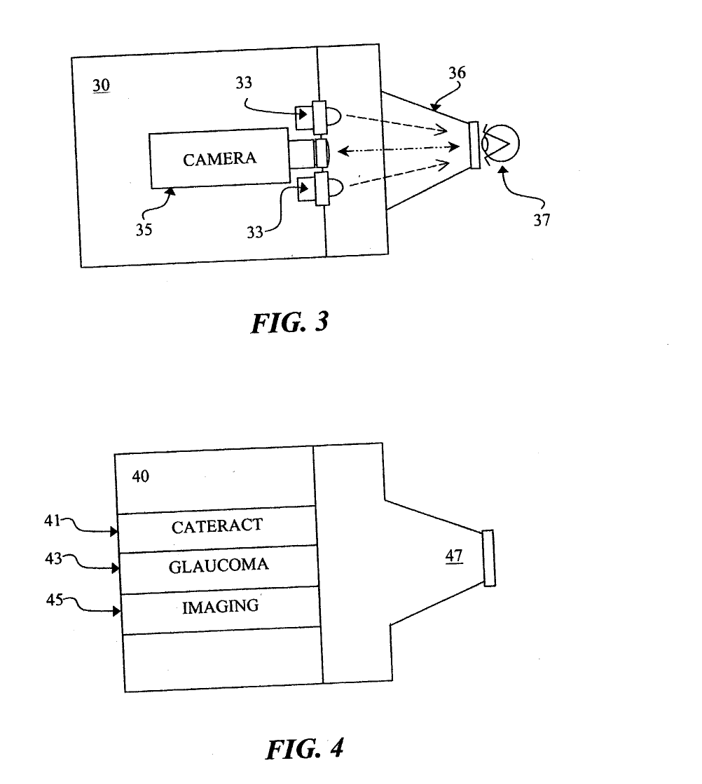

Referring to FIG. 4, another embodiment of the present invention is illustrated wherein cataract imaging technology 41 as described herein can be incorporated in combination with known glaucoma screening technology 42. Imaging 45 resources can are also provided in the combined system to record analysis of the patient. Latest glaucoma assessment means are laser based and can assess a patient eye at the nerve level. It should be appreciated given the present teaching that cataract screening can be combined with current assessment in order to economize in terms of time and money. With a combined glaucoma/cataract apparatus, a patient may only have to be captive in front of equipment once. Practitioners on the other hand can conserve space and equipment costs with a dual-use ocular assessment apparatus.

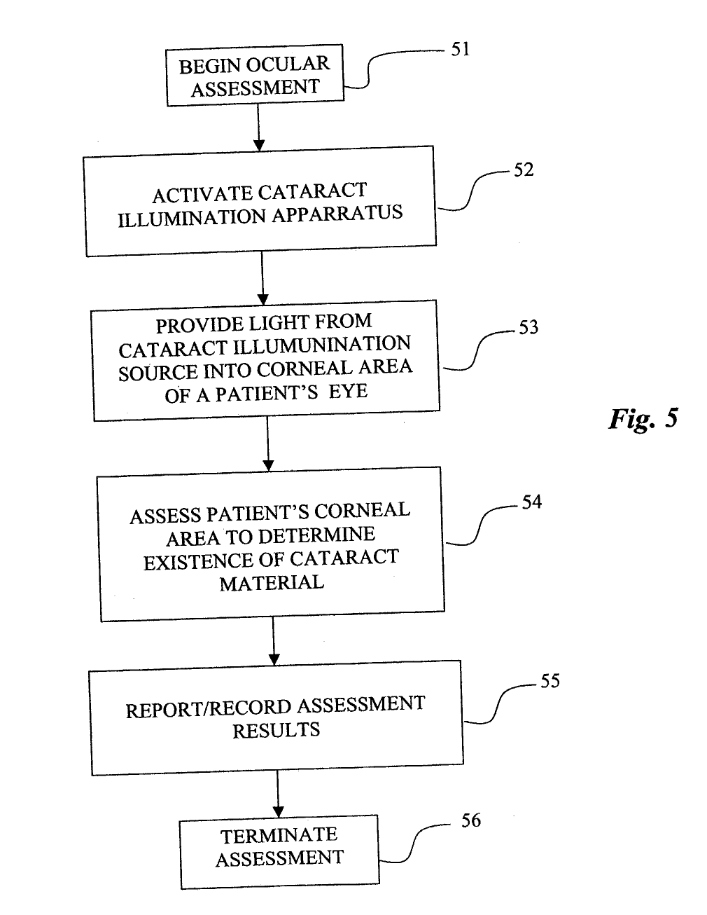

Referring to FIG. 5, one method according to preferred embodiments of the present invention will now be described. Ocular assessment is initiated in step 51 and can include preparing a patient for assessment. As seen in block 52, the cataract illumination apparatus is activated 52 once a patient is readied. In block 53, light is provided from the illumination source into the corneal area of a patient's eye. In block 54, once illuminated, the corneal area of the patient's eye is assessed to determine if cataract material exists. Assessment may be through imaging (camera) or directly by a practitioner or assistant. Once an assessment is completed, a report or record of screening results is rendered as shown in block 55. Report may be by communication directly to the patient, or can be in the form of imaging results (digital image/file). In block 56, after report/record of results 55, assessment is terminated.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

We claim:

1. A method of corneal area analysis, comprising the steps of:
providing blue light from a light source into the corneal area of a patient's eye; and
assessing the illuminated corneal area of a patient's eye to detect the presence of cataract material.

2. The method of claim 1, further comprising reporting the results of said assessment.

3. The method of claim 1, further comprising recording the results of said assessment.

4. The method of claim 3 wherein said recording is by digital imaging of said illumination of said corneal area.

5. The method of claim 1 further comprising recording said illumination of said corneal area using digital imaging.

6. A method of performing corneal analysis on a patient's eye, comprising the steps of:
providing blue light from a light source into the corneal area of a patient's eye;
recording illumination of said corneal area of said patient's eye; and
assessing recorded illumination of said corneal area of a patient's eye to detecting the presence of cataract material.

7. The method of claim 6, further comprising reporting the results of said assessment.

8. The method of claim 6 wherein said recording illumination of said corneal area of said patient's eye is by digital imaging.

9. Apparatus for analyzing the corneal area of a patient's eye, comprising illumination source for providing blue light into the corneal area of a patient's eye.

10. The apparatus of claim 9, further comprising a camera for recording illumination of the corneal area of a patient's eye.

11. The apparatus of claim 9 wherein said apparatus is a portable hand held device.

12. The apparatus of claim 9 further comprising a glaucoma assessment means.

13. An apparatus for assisting an evaluation of a disease in an eye of a patient, comprising:
blue light illumination source for illuminating the corneal area of a patient's eye;
imaging means for recording illumination of a patient's eye; and
imaging area for providing a controlled environment for illumination and imaging of the corneal area of a patient's eye.

14. The apparatus of claim 13 further comprising a glaucoma assessment means.
